(19) United States
(12) Patent Application Publication
(10) Pub. No.: US 2008/0255411 A1
(43) Pub. Date: Oct. 16, 2008

(54) TOOL FOR ENDOSCOPE AND ENDOSCOPE SYSTEM

(75) Inventor: Shotaro KOBAYASHI, Saitama (JP)

Correspondence Address: GREENBLUM & BERNSTEIN, P.L.C., 1950 ROLAND CLARKE PLACE, RESTON, VA, 20191, US

(73) Assignee: C/O PENTAX CORPORATION, Tokyo (JP)

(21) Appl. No.: 11/867,043

(22) Filed: Oct. 4, 2007

(30) Foreign Application Priority Data
Oct. 5, 2006 (JP) 2006-274052

(51) Int. Cl. A61B 1/06

(52) U.S. Cl. 600/104; 600/248

(57) ABSTRACT

An endoscope system includes a light source, a scope, a detector, an adjuster, and a tool. The light source emits illuminating light to illuminate a subject. The scope transmits the illuminating light for emission from the end of the scope. The detector detects the luminance and/or chromaticity of an entering light that enters the scope. The adjuster adjusts the light amount and/or white balance of the illuminating light, based on the luminance and/or chromaticity of the entering light. The end of the scope is inserted into the tool. The tool includes a reflecting member that reflects the illuminating light emitted from the end of the scope so as to become the entering light. The distance between the end of the scope that is inserted into the tool and the reflecting member is adjustable.

### BACKGROUND OF THE INVENTION

1. Field of the Invention

The present invention relates to a tool for an endoscope and to an endoscope system, and especially, to a tool for optical control of an endoscope, and to an endoscope system in which optical control is carried out.

2. Description of the Related Art

A light source is provided in the processor of an endoscopic device. Illuminating light emitted by a light source is transmitted via a scope, and is used for illuminating a subject such as an intracorporeal organ. Whether the amount of the illuminating light emitted by the light source is at a predetermined level or not is generally checked at the time of the production of the endoscopic device and other times, by connecting a luminance meter to the case of the processor.

Also, it is known that the amount of illuminating light is adjusted so that the illuminating light having suitable amount for white-balance adjustment is emitted from the end of the scope.

Generally, the amount of the illuminating light emitted by the light source decreases with long term usage. Therefore, even though the amount of illuminating light emitted by the light source has been checked at the time of the production of the endoscopic device, the actual amount of illuminating light emitted by the light source may become unsuitable. Thus, the ability to adjust the amount of illuminating light to compensate for the decrease expected on the basis of usage time of the light source is a consideration. However, this method is not infallible, because light sources of the same type may have different trends in their weakening over time, or other irregularities.

When the amount of illuminating light emitted by the light source is inadequate, it is difficult to adjust the amount of illuminating light emitted from the scope accurately.

### SUMMARY OF THE INVENTION

Therefore, an object of the present invention is to provide a tool that is used with an endoscope to adjust the amount of illuminating light accurately, and an endoscope system that enables accurate adjustment of the amount of illuminating light.

An endoscope system according to the present invention includes a light source, a scope, a detector, an adjuster, and a tool. The light source emits illuminating light to illuminate a subject. The scope transmits the illuminating light for emission from an end of the scope. The detector detects the luminance and/or chromaticity of an entering light that enters the scope. The adjuster adjusts the light amount and/or white balance of the illuminating light based on the luminance and/or chromaticity of the entering light. The end of the scope is inserted into the tool. The tool includes a reflecting member that reflects the illuminating light emitted from the end of the scope so as to become the entering light. The distance between the end of the scope that is inserted into the tool and the reflecting member is adjustable.

The tool may further include a distance detector that detects the distance, and a distance adjuster that adjusts the distance. The adjuster may adjust the light amount of the illuminating light to a target amount.

The endoscope system may further include a memory in which the data representing a target distance that is a target value of the distance between the end of the scope that is inserted in the tool and the reflecting member, is stored.

In the endoscope system, one of a plurality of the scopes may be selectively used, and the endoscope system may further include an identifier that identifies the scope that is in use.

In the endoscope system, one of a plurality of the scopes may be selectively used, and the target distance that is a target value of the distance between the end of the scope that is inserted into the tool and the reflecting member may be set for each of the scopes.

The endoscope system may further include a processor to which the scope is connected, and the tool may be provided in the processor.

The adjuster may include a focusing lens that is movable in the direction of the optical axis thereof. The endoscope system may further include an adjustment commander that commands the adjuster to adjust the light amount and white balance of the illuminating light in a single operation.

A tool according to the present invention is used with an endoscope in which an illuminating light emitted by a light source is transmitted to a subject by a scope, the luminance and/or chromaticity of an entering light that enters the scope is detected, the light amount and/or white balance of the illuminating light is adjustable based on the luminance and/or chromaticity of the entering light. The tool includes an attachment to which the scope is attached, and a reflecting member that reflects the illuminating light emitted from an end of the scope so as to become the entering light. The distance between the end of the scope that is attached to the attachment and the reflecting member is adjustable.

The tool may further include a moving member that moves at least one of the attachment and the reflecting member to adjust a distance between the end of the scope and the reflecting member.

The tool may further include a receiver that receives data representing a target distance that is a target value of the distance between the end of the scope that is attached to the attachment and the reflecting member, and that is transmitted by the endoscope.

The tool may further include a mark that represents relative position of the attachment with respect to the reflecting member, and that is provided on the attachment or the reflecting member.

### BRIEF DESCRIPTION OF THE DRAWINGS

The present invention will be better understood from the description of the preferred embodiments of the invention set forth below, together with the accompanying drawings, in which:

FIG. 1 is a block diagram of an endoscopic device of a first embodiment;

FIG. 2 shows a distance sensor of the endoscopic device;

FIGS. 3A to 3C show strengths of the light received by the distance sensor;

FIG. 4 is a flow chart representing a light-amount and white-balance adjustment routine in the endoscopic device;

FIG. 5 is a flow chart representing a light-amount adjustment routine that is a part of the light-amount and white-balance adjustment routine;

FIG. 6 is a flow chart representing a white-balance adjustment routine that is a part of the light-amount and white-balance adjustment routine;

FIG. 7 is a block diagram of an endoscope system of a second embodiment;

FIG. 8 is a block diagram of an endoscope system of a third embodiment; and

FIG. 9 is a block diagram of a current endoscopic device.

### DESCRIPTION OF THE PREFERRED EMBODIMENTS

Hereinafter, the preferred embodiments of the present invention are described with reference to the attached drawings.

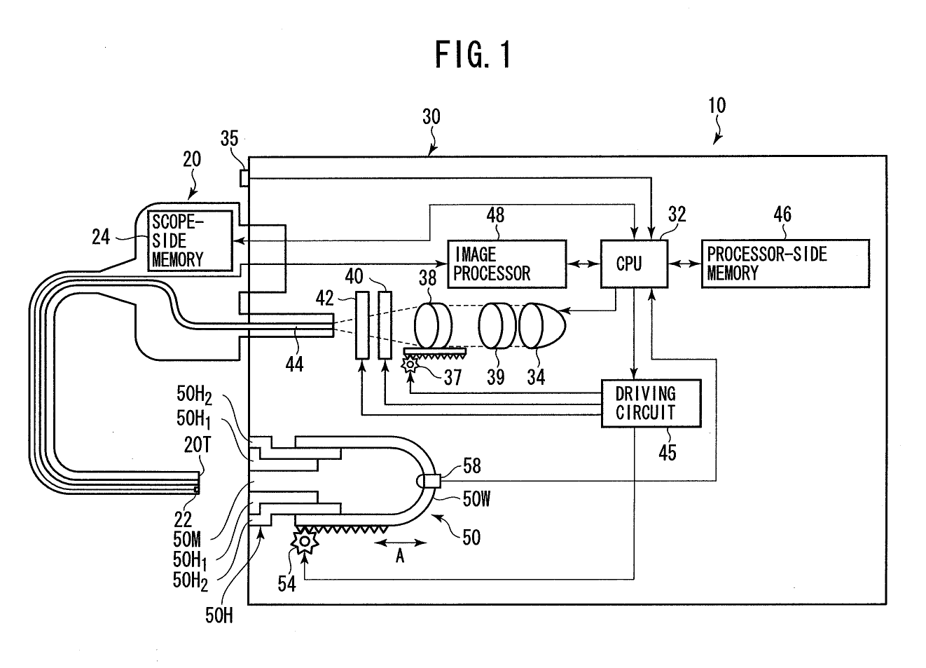

As shown in FIG. 1, an endoscopic device (or an endoscope system) 10 includes a scope 20 and a processor 30. The scope 20 transmits illuminating light to a subject, and generates image signals on the basis of reflected light from the subject. The processor 30 processes the image signals transmitted from the scope 20. In the endoscopic device 10, one of a plurality of scopes including the scope 20 is selected, and is detachably connected to the processor 30 for usage. A keyboard for user input of commands, and a monitor to display a subject image (both not shown) are connected to the processor 30.

In the processor 30, a CPU 32 for controlling the entirety of the processor 30, a light source 34 to emit illuminating light, and other components are provided. When the processor starts, the light source 34 emits the illuminating light, under the control of the CPU 32. The emitted illuminating light is made parallel by a collimator lens 39. The amount of the illuminating light is adjusted by a focusing lens 38 and an aperture 40. The illuminating light passes through a rotary shutter 42, and enters a light guide 44. The illuminating light which has passed through the light guide 44 is emitted toward a body cavity as a subject from an end 20T of the scope 20.

The illuminating light reflected by the subject reaches a light-receiving surface of a CCD (or a detector) 22, that is provided near the end 20T of the scope 20. As a result, image signals are generated by the CCD 22. The endoscopic device 10 is of the simultaneous type. Image signals generated by the CCD 22 are read successively frame by frame, by a process circuit (not shown) provided in the scope 20, and transmitted to an image processor 48.

Predetermined processes such as white balance adjustment are carried out on the image signals transmitted to the image processor 48. Then, luminance signals and color-difference signals are generated, and are stored in a processor-side memory 46 after the analog to digital conversion and other processes. Further, luminance signals and color-difference signals are read from the processor-side memory 46, under the control of the CPU 32, and are output to the monitor after predetermined processes. As a result, a moving image of the subject is displayed on the monitor as a real-time image.

In the scope 20, a scope-side memory 24 is provided. Information identifying the scope 20, and data for signal processing in the scope 20, such as white balance adjustment, are previously stored in the scope-side memory 24. When the scope 20 is connected to the processor 30, the information identifying the scope 20 is read by the CPU 32. Therefore, the scope selected from a plurality of scopes compatible with the processor 30, and currently in use, is identified as the scope 20.

In the processor 30, a tool 50 in which the end 20T of the scope 20 is inserted, is provided. The tool 50 is provided to accurately adjust the amount of the illuminating light emitted by the light source 34 and the white balance, as explained below.

The tool 50 includes a holder (or an attachment) 50H and a reflecting cup 50W whose end is nearly spherical. The end 20T of the scope 20 inserted into the mouth 50M of the tool 50, is attached and held in a predetermined position by the holder 50H with suitable friction. The inner surface of the reflecting cup (or a reflecting member) 50W is white, and is somewhat rough. Therefore, illuminating light emitted from the end 20T of the scope 20 is reflected and diffused by the inner surface of the reflecting cup 50W. While the end 20T is inserted into the mouth 50M, no light other than the illuminating light enters the tool 50 from outside, because the mouth 50M is closed by the end 20T of the scope 20.

The holder 50H includes an inner cylinder 50H₁ that contacts the outer surface of the end 20T of the scope 20, and an outer cylinder 50H₂ that contacts the inner surface of the reflecting cup 50W. The inner cylinder 50H₁ and the outer cylinder 50H₂ are fixed. A small-diameter part of the outer cylinder 50H₂ with a diameter smaller than other part thereof, is fit into the inside of the reflecting cup 50W, so as to be slidable and have suitable friction against the cup 50W. Thus, the holder 50H and the reflecting cup 50W are slidable against each other.

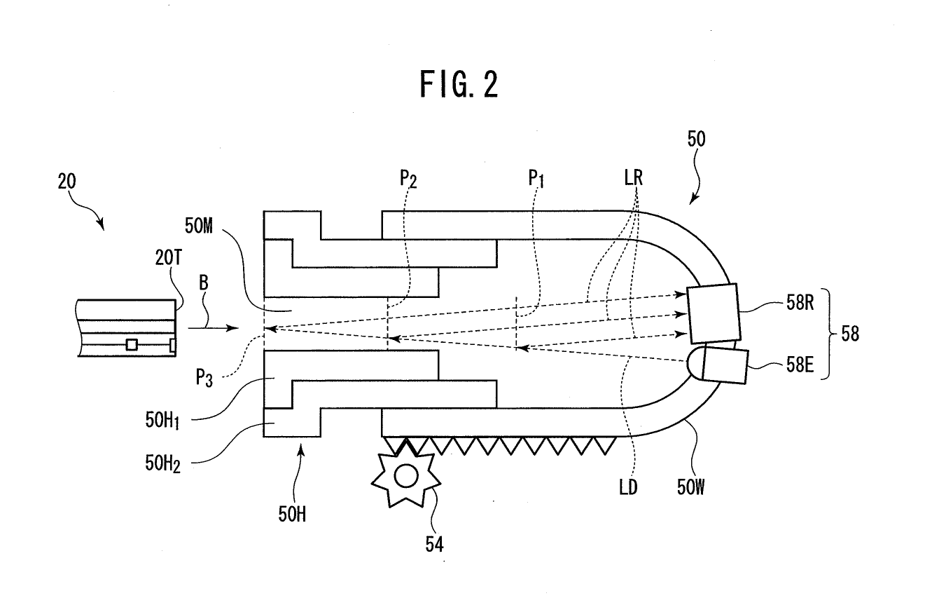

In the tool 50, a distance sensor (or a distance detector) 58 that detects the distance between the end 20T of the scope 20 that is inserted in a predetermined position and the reflecting cup 50W, and a tool motor (or a distance adjuster, or a moving member) 54 that moves the reflecting cup 50W, are provided. Target-distance data, that represents a target value of the distance (a target distance) between the end 20T of the inserted scope 20 and the reflecting cup 50W, is previously stored in the processor-side memory 46. The target distance value is set for each of the scopes compatible with the endoscopic device 10. The distance sensor 58 includes a light-emitting part 58E that emits a detection light LD, and a light-receiving part 58R (see FIG. 2) that receives reflected light LR that is a reflected light of the detection light LD and that is reflected by the object of the distance measurement. The light-receiving part 58R comprises a PSD (Position Sensitive Detector), for example. In this case, the distance sensor 58 detects the distance from the end 20T of the scope 20 to the distance sensor 58, based on the received position of the received reflected light LR on the light receiving part 58R.

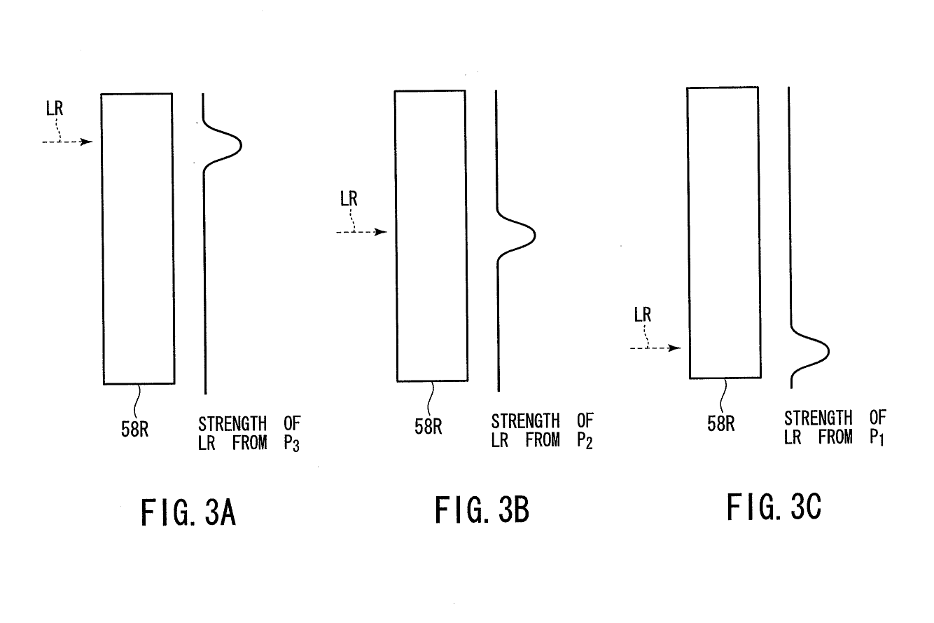

For example, the location of the reflected light LR peak on the receiving part 58R depends on which of the positions P₁ to P₃ the end 20T is at as shown in FIGS. 3A to 3C. Therefore, the distance from the end 20T of the inserted scope 20 to the distance sensor 58 can be detected on the basis of the peak of the reflected light LR on the receiving part 58R.

An adjustment switch 35 for adjusting the light amount and white balance is provided, on the surface of the processor 30. When the adjustment switch 35 is depressed and is turned on, command signals for adjustment are transmitted to the CPU 32. As a result, the amount of the illuminating light and white balance are adjusted as explained below.

First, since the scope in use has already been identified as scope 20, the data representing the target distance value for the scope 20 is read from the processor-side memory 46 by the CPU 32. The CPU 32 controls a driving circuit 45 so that the distance between the end 20T of the scope 20 and the reflecting cup 50W detected by the distance sensor 58, matches the target distance represented by the read data. To achieve this, the tool motor 54 moves the reflecting cup 50W in a direction which is indicated by an arrow A and which is parallel to the insertion direction of the scope 20, under the control of the driving circuit 45.

After the distance between the end 20T of the scope 20 and the reflecting cup 50W has been adjusted as explained above, the illuminating light is emitted from the end 20T of the scope 20. The illuminating light reflected by the reflecting cup 50W enters the CCD 22 as an entering light. Because the entering light is diffused by the reflecting cup 50W, the entering light enters the CCD 22 as white light having constant brightness and without unevenness. The amount of the illuminating light and white balance are adjusted based on the luminance signals and color-difference signals generated in the image processor 48 by the entering light entered the CCD 22. That is, the amount of the illuminating light and white balance are adjusted based on the luminance and chromaticity of the entering light detected by the CPU (or a detector) 32.

The data representing the target value for the amount of the illuminating light (a target light amount) used for illuminating a subject is previously stored as a luminance signal value in the processor-side memory 46. The data of the target value for the illuminating light amount for the identified scope 20 is also read by the CPU 32, in a manner similar to the reading of the data of the target distance value. When the actual amount of the illuminating light entering the light guide 44 is not equal to the target value of the illuminating light amount, the CPU 32 controls the driving circuit 45 so that a lens motor 37 is driven by the driving circuit 45 and the position of the focusing lens 38 is adjusted along its optical axis.

As a result, the amount of the illuminating light entering the light guide 44 is adjusted to match the target value as explained below. Note that, in the light amount adjustment, the aperture 40 has been previously set by the driving circuit 45 to a fully-opened position. This is to prevent an error in the light amount adjustment caused by a variability in the opening and closing positions of the aperture 40. Thus, the amount of the illuminating light is accurately adjusted by using only the focusing lens 38.

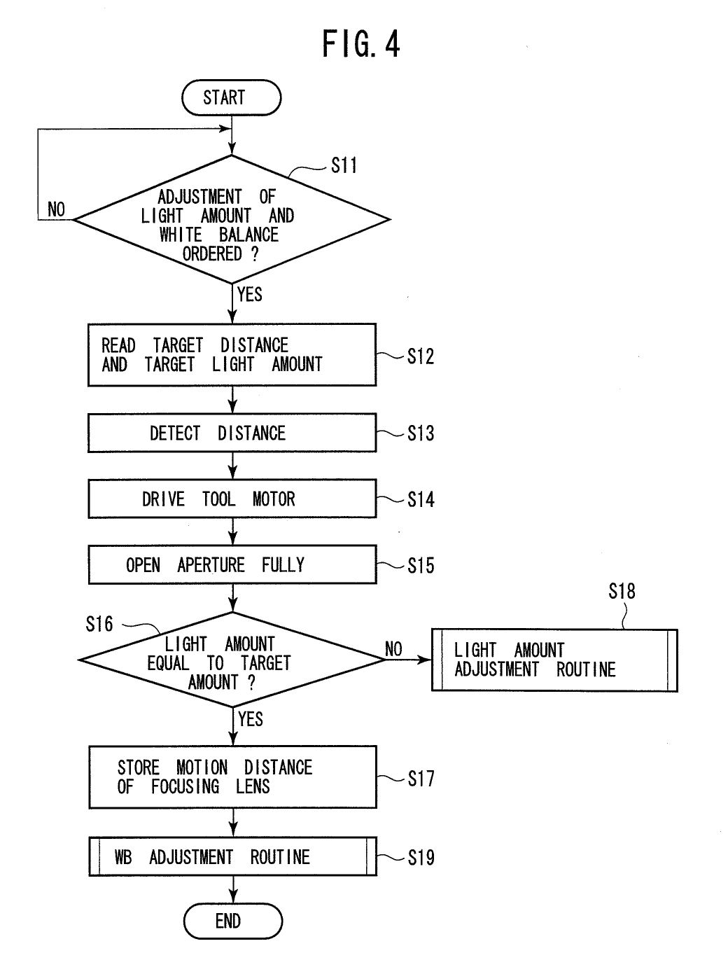

The light-amount and white-balance adjustment routine (see FIG. 4) starts when the scope 20 is connected to the processor 30 and the end 20T of the scope 20 is inserted into the mouth 50M of the tool 50. At step S11, it is determined whether the adjustment switch 35 is depressed or not; that is, it is determined whether light mount and white balance adjustment has been ordered. When it is determined that light mount and white balance adjustment has been ordered, the process proceeds to step S12. At step S12, the data of the target distance value and the target illuminating light amount for the identified scope 20 being used in the endoscopic device 10, are read, and the process proceeds to step S13.

At step S13, the distance between the end 20T of the scope 20 and the reflecting cup 50W is detected by the distance sensor 58, and the process proceeds to step S14. At step S14, to make the distance between the end 20T of the scope 20 and the reflecting cup 50W be equal to the target distance value, the tool motor 54 is driven to move the reflecting cup 50W. Then the process proceeds to step S15. At step S15, the aperture 40 is set fully open, and the process proceeds to step S16.

At step S16, it is determined whether the actual amount of the illuminating light is equal to the target amount or not; that is, it is determined whether the value of the luminance signal generated by the entering light is equal to the set target luminance value. When it is determined that the actual amount of the illuminating light is equal to the target amount, the process proceeds to step S17, and when it is determined that the actual amount is different from the target amount, the process proceeds to step S18.

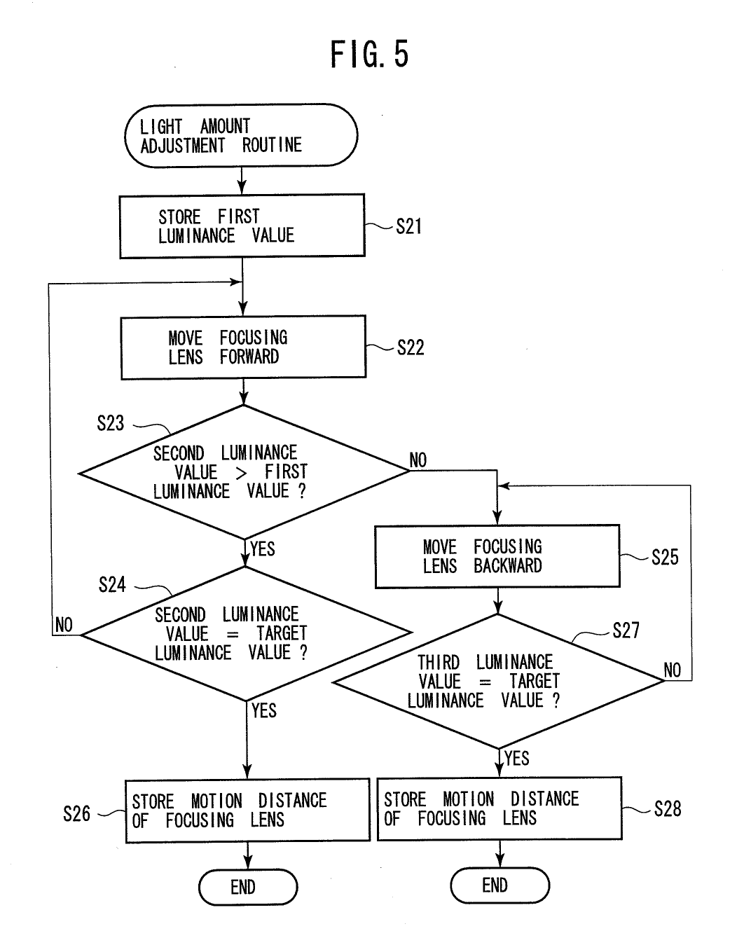

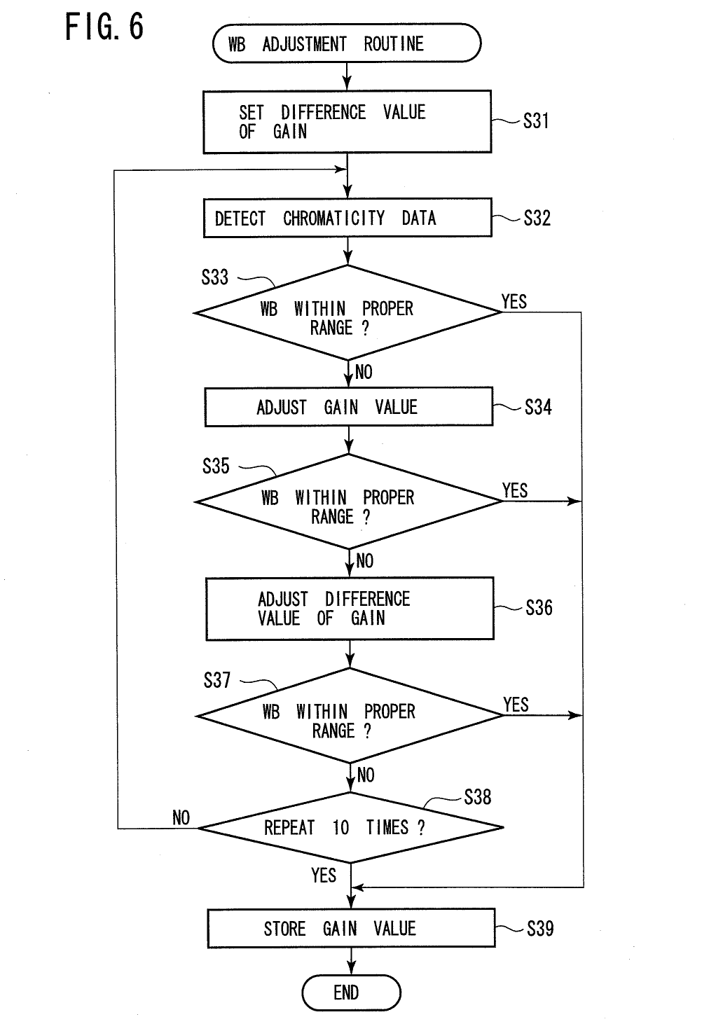

At step S17, data of the motion distance of the focusing lens 38, as data useful for subsequent light amount adjustments, is stored in the processor-side memory 46. Then, the process proceeds to step S19. At step S18, the light-amount adjustment routine (see FIG. 5) is carried out, and at step S19, the white-balance adjustment routine (see FIG. 6) is carried out.

When the light-amount adjustment routine starts, at step S21 (see FIG. 5), the value of the luminance signal generated by the entering light is obtained, and the data of the obtained luminance value is stored as a first luminance value, in the luminance value memory (not shown) of the processor-side memory 46 (see FIG. 1). Then the process proceeds to step S22. At step S22, the focusing lens 38 is moved forward to change the amount of the illuminating light passing through the focusing lens 38, and then the process proceeds to step S23.

At step S23, it is determined whether a second luminance value, (the value of the luminance signal generated by the reflected illuminating light that passes through the forwardly moved focusing lens 38) is larger than the first luminance value stored in the luminance value memory, or not. When it is determined that the second luminance value is larger than the first luminance value, the process proceeds to step S24, and when it is determined that the second luminance value is equal to or smaller than the first luminance value, the process proceeds to step S25.

At step S24, it is determined whether the second luminance value is equal to the target luminance value. When it is determined that the second luminance value is equal to the target luminance value, the process proceeds to step S26, and when it is determined that the second luminance value is different from the target luminance value, the process returns to step S22.

At step S26, data representing the motion distance of the focusing lens 38, that is, the motion distance from the position of the focusing lens 38 prior to the light amount adjustment, to the position where the second luminance value matches the target luminance value is obtained, is stored in the processor-side memory 46. This data is useful for subsequent light amount adjustments. Then, the light-amount adjustment routine ends, and the process proceeds to step S19 (see FIG. 4) in the light amount and white-balance adjustment routine.

At step S25, the focusing lens 38 is moved backwards, and the process proceeds to step S27. At step S27, it is determined whether a third luminance value (the value of the luminance signal generated by the reflected illuminating light that is passed through the backwardly moved focusing lens 38) is equal to the target luminance value. When it is determined that the third luminance value is equal to the target luminance value, the process proceeds to step S28, and when it is determined that the third luminance value differs from the target luminance value, the process returns to step S25.

At step S28, similarly to step S26, data representing the motion distance of the focusing lens 38, that is, the motion distance from the position of the focusing lens 38 prior to the light amount adjustment to its final position, is stored in the processor-side memory 46. Then, the light-amount adjustment routine ends, the process proceeds to step S19 (see FIG. 4), and the white-balance adjustment routine (see FIG. 6) starts.

In the white-balance adjustment routine, white balance is adjusted by operations in the CPU 32, as explained below. Here, the G (green) gain is fixed, and the B (blue) and R (red) gains are adjusted, so that white balance is adjusted. At step S31, the values of R and B gains are read from the scope-side memory 24, and the predetermined difference values of the R and B gains, used for the white balance adjustment explained below, are set. Then the process proceeds to step S32.

At step S32, chromaticity data is detected based on the image signals of two fields generated by the CCD 22 from the entering light, that is, the illuminating light reflected by the inner surface of the white reflecting cup 50W of the tool 50 and entered the CCD 22. Then, the process proceeds to step S33. At step S33, based on the detected chromaticity data, it is determined whether the current white balance is within the proper range previously set. When it is determined that the current white balance is out of the proper range, and white balance adjustment is thus required, the process proceeds to step S34. When it is determined that the current white balance is within the proper range, and no adjustment is required, the process proceeds to step S39.

At step S34, both R and B gain values are adjusted by adding the difference values set at step S31, then the process proceeds to step S35. At step S35, it is determined whether the white balance adjusted by the changed values of R and B gains is within the proper range that is previously set, that is, it is determined whether further white balance adjustment is required. When it is determined that the white balance is out of the proper range and further adjustment is required, the process proceeds to step S36, but when it is determined that the white balance is within the proper range and no more adjustment is required, the process proceeds to step S39.

At step S36, difference values are adjusted. That is, new difference values having smaller absolute values than the corresponding difference values previously set at step S31 or step S36 of the previous process cycle, for each of the values of R and B gains, are set. Then the process proceeds to step S37. At step S37, it is again determined whether the white balance is within the proper range previously set. When it is determined that the white balance is out of the proper range and further adjustment is required, the process proceeds to step S38, and when it is determined that the white balance is within the proper range and no more adjustment is required, the process proceeds to step S39.

At step S38, it is determined whether steps S32 to S37 have been repeated 10 times. When it is determined that steps S32 to S37 have been repeated 10 times, the process proceeds to step S39, without any further adjustment. This is because, by then, the white balance should have reached a near optimal level. On the other hand, when it is determined that steps S32 to S37 have not been repeated 10 times, the process returns to step S32, and further white balance adjustment is carried out. At step S39, the final values of R and B gains are stored in the scope-side memory 24, as useful data for subsequent white balance adjustment. Then, the white-balance adjustment routine ends.

As explained above, in the first embodiment, the amount of the illuminating light used to illuminate a subject can be adjusted to the target amount set for each of the scopes, even though the amount of the illuminating light emitted by the light source 34 may have drifted from its original level due to extended use of the light source 34 or other reasons. For the light amount adjustment, the entering light that is the reflected illuminating light reflected by the tool 50 under the constant condition is used every time, so that precise adjustment of the amount of the illuminating light is possible. Furthermore, in terms of the white balance adjustment, using the tool 50 can prevent a halation, and reflected light to become the entering light having suitable luminance and chromaticity for white balance adjustment can always be obtained. Thus, white balance can be accurately adjusted.

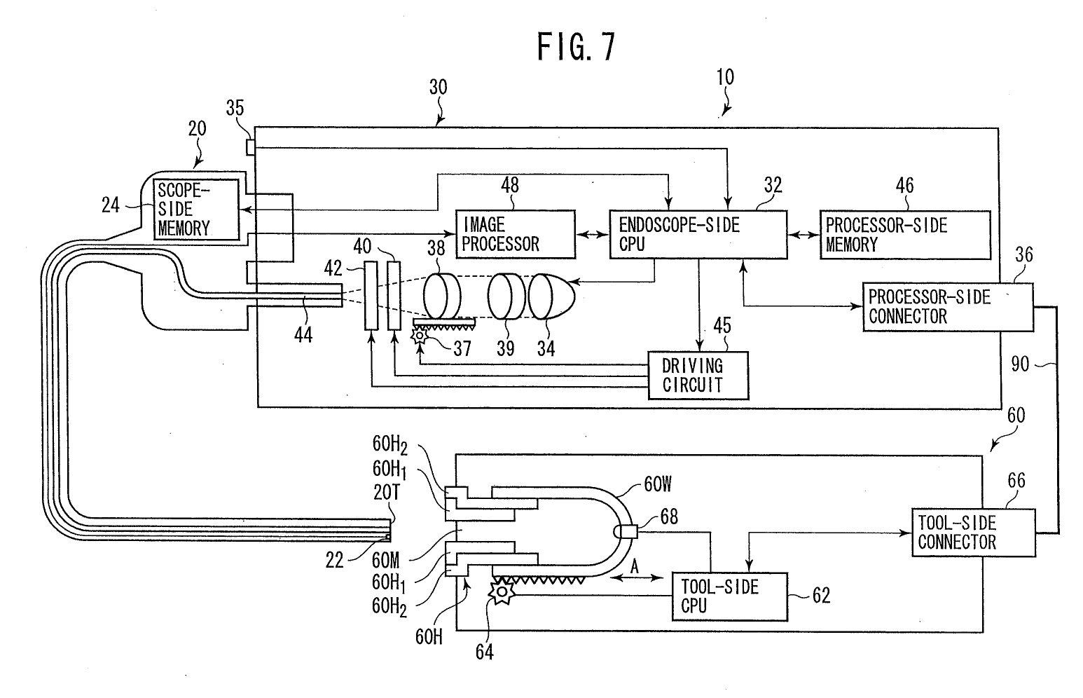

Next, the second embodiment and the main differences between it and the first embodiment are explained. Note that in FIG. 7, the corresponding components to those of the first embodiment are identified by the same numerals, except for those included in a tool 60.

The endoscopic device 10 of the present embodiment has the following differences from that in the first embodiment, where the tool 60 is an independent apparatus of the processor 30, and the tool 60 is detachably attached to the processor 30. When the tool 60 is attached to the endoscopic device 10, a tool-side connector 66, and a processor-side connector 36 provided in the endoscopic device 10, are connected to each other through a connecting cable 90.

A tool-side CPU 62 is provided in the tool 60. The tool-side CPU 62 controls the entirety of the tool 60, and communicates with the endoscope-side CPU 32 that is electrically connected to the tool-side CPU 62 via the processor-side connector 36 and tool-side connector 66.

Thus, when the scope in use is identified as the scope 20 by the endoscope-side CPU 32, and the target distance value of the scope 20 is read from the processor-side memory 46, signals representing the target distance value are transmitted to the tool-side CPU 62 from the endoscope-side CPU 32, via the processor-side connector 36 and tool-side connector 66.

The tool-side CPU (or a receiver) 62 receives the information on the target distance value, and controls a tool motor (or a distance adjuster) 64, so that the distance detected by a distance sensor (or a distance detector) 68 between end 20T of the scope 20 held by a holder (or an attachment) 60H and a reflecting cup 60W, is adjusted to equal the target distance. At that time, the reflecting cup 60W is moved in a direction indicated by an arrow A and parallel to the insertion direction of the scope 20 to a mouth 60M. After that, the illuminating light is emitted from the end 20T of the scope 20, and the illuminating light reflected by the reflecting cup (or a reflecting member) 60W enters the CCD 22 as the entering light.

Thereafter, similarly to the first embodiment, the amount of the illuminating light is adjusted under control of the endoscope-side CPU 32, and the white balance is also adjusted based on the chromaticity of the entering light detected by the endoscope-side CPU 32.

As explained above, in the second embodiment, the tool 60 can be used with the conventional endoscopic device 10, including the processor 30 in which the tool 60 is not provided, by storing the required information such as the data representing the target distance value and target amount of the illuminating light for the scope 20 in the endoscope-side CPU 32. As a result, the amount of the illuminating light and white balance can be accurately adjusted by simple operations.

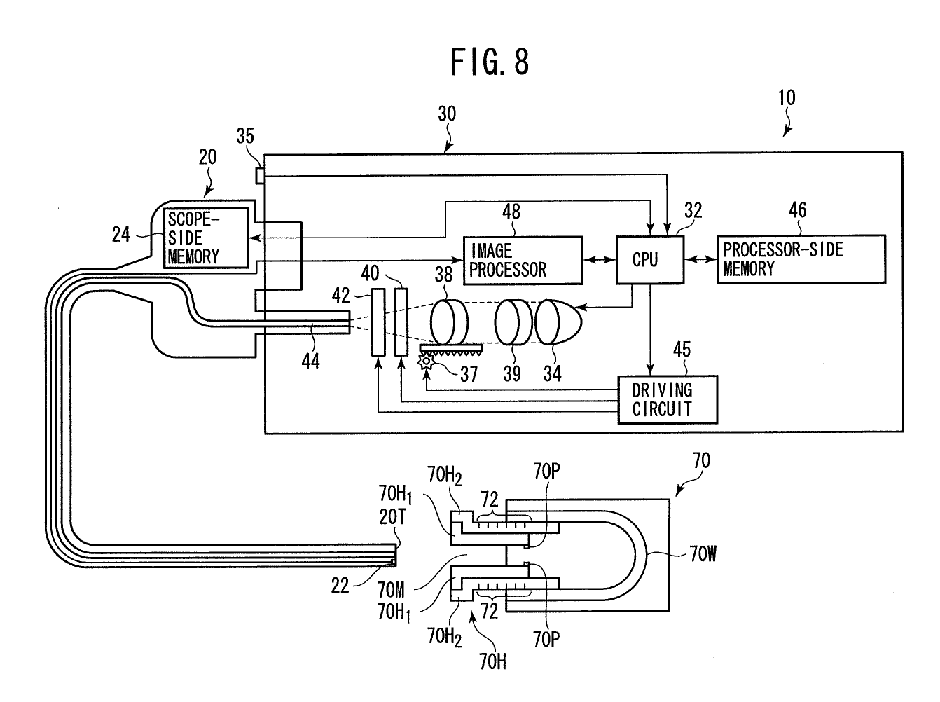

Next, the third embodiment and the main differences between it and the above described embodiments are explained. Note that in FIG. 8, corresponding components to those of the first and second embodiments are identified by the same numerals, except for those included in a tool 70.

The endoscopic device 10 of the present embodiment has the following differences from that in the second embodiment, where the tool 70 is not connected to the processor 30 electrically, and a part of the operations which are carried out automatically in the second embodiment, are instead carried out by the user. That is, in the tool 70, the tool-side CPU 62, the tool motor 64, the distance sensor 68, and other components are not provided, and a mark for adjustments of the distance between the end 20T of the scope 20 and the reflecting cup 70W the below explained, is provided on the surface of the tool 70.

Just as in the former embodiments, a holder 70H of the present embodiment includes an inner cylinder 70H₁ that contacts the outer surface of the end 20T of the scope 20, and an outer cylinder 70H₂ that is fixed to the outer surface of the inner cylinder 70H₁, where the inner cylinder 70H₁ and the outer cylinder 70H₂ are slidable against each other. However, the sliding operation between the inner and outer cylinders 70H₁ and 70H₂ is carried out by the user, unlike in the previous embodiments.

Mark 72 has scales provided on the outer surface of the outer cylinder 70H₂, as illustrated in FIG. 8. Using the mark 72, the user can visually confirm the relative position with respect to the holder 70H that is a slidable member to the reflecting cup 70W that is the other slidable member. That is, the user can visually and easily confirm the protruding distance of the holder 70H from the reflecting cup 70W, and thereby, the distance between the inner cylinder 70H₁ in the holder 70H, and the reflecting cup 70W.

Projections 70P are provided at the end of the inner surface of the inner cylinder 70H₁. Therefore, the end 20T of the scope 20 inserted into the mouth 70M, is always attached in the position where the end 20T contacts the projections 70P. As a result, distance-measurement errors caused by the insertion operation of the end 20T can be prevented, because the distance between the inner cylinder 70H₁ and the reflecting cup 70W, that is, the distance between the end 20T of the scope 20 and the reflecting cup 70W are always constant. Furthermore, unlike the previous embodiments, although the distance between the end 20T and the reflecting cup 70W cannot automatically be adjusted, it can be prevented that the end 20T of the scope 20 that is inserted by a wrong operation by the user, reaches and breaks the reflecting cup 70W, because of the projections 70P.

Note that in the mark 72, in addition to or instead of the scales with equal intervals, a sign (not shown) representing the insertion position of the scope 20 required to achieve the target distance between the end 20T and the reflecting cup 70W, may be provided. In this case, the distance can easily be adjusted by a sliding operation of the holder 70H along the reflecting cup 70W by reaching the marked position, so that the ease of use of the tool 70 increases. Note that the signs are preferably provided for all of the scopes compatible with the endoscopic device 10 including the scope 20. The mark 72 may be provided on the reflecting cup 70W, as long as the mark 72 is visible from the outer of the tool 70.

As explained above, in the third embodiment, as well as the second embodiment, the tool 70 can be used with the conventional endoscope 10. Further, the structure of the processor 30 can be simplified, because neither the tool 70 nor the processor-side connector 36 (see FIG. 7) is required.

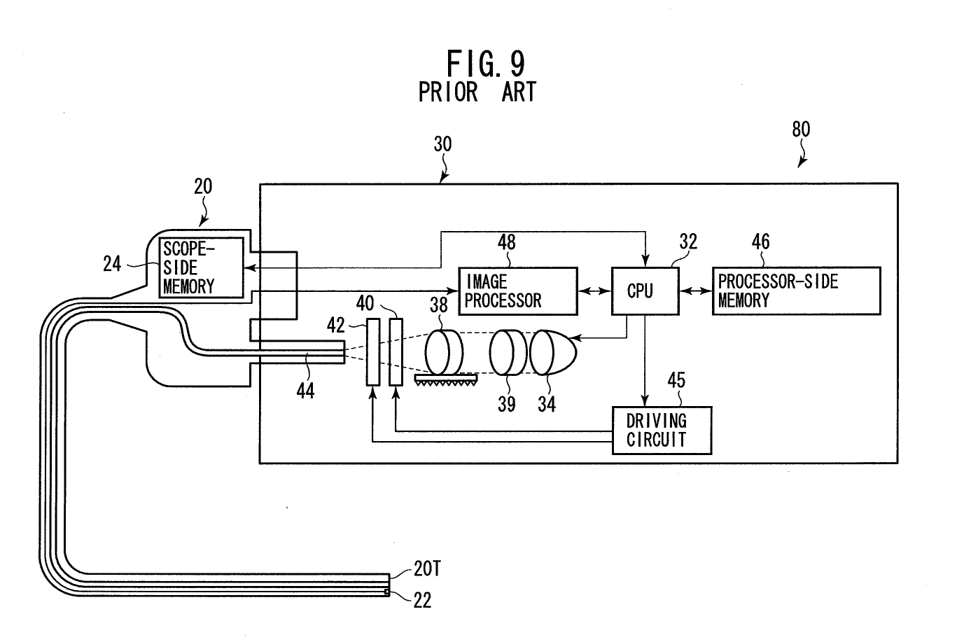

On the other hand, in a conventional endoscopic device 80 (see FIG. 9), the tool 50, 60, and 70 explained in the above embodiments are not provided and is not connected. Therefore, accurately adjusting the illuminating light emitted by the light source 34 is difficult. Further, when a conventional tool other than the tools 50, 60, and 70 is used for adjusting the white balance, accurate white balance adjustment is difficult. This is because illuminating light whose amount is suitable for the white balance adjustment cannot used, so that halation may occur due to excessive light and may prevent accurate adjustment of the white balance.

Note that the configurations of the endoscopic device 10 including the tool 50, 60 or 70, and so on are not limited to the aforementioned embodiments. For example, in the tools 50, 60, and 70, an adapter may be used to enable the insertion of a plurality of scopes having different diameters. Further, although it is preferable to provide the adjustment switch 35 to adjust both the light amount and white balance in a single operation, one switch for adjusting the light amount, and a separate one for adjusting the white balance may be provided on the processor 30.

A message or the like to remind the user to carry out the light amount adjustment by using the tool 50, 60, or 70 may be displayed on the monitor, at a suitable time, such as when the light source 34 is exchanged, or when the accumulated usage time of the light source 34 exceeds a predetermined time limit. Further, the data of the target distance values and the target light amounts may be input or updated externally, such as with a keyboard.

The invention is not limited to that described in the preferred embodiment; namely, various improvements and changes may be made to the present invention without departing from the spirit and scope thereof.

The present disclosure relates to subject matter contained in Japanese Patent Application No. 2006-274052 (filed on Oct. 5, 2006) which is expressly incorporated herein, by reference, in its entirety.

1. An endoscope system comprising:
a light source that emits illuminating light to illuminate a subject;
a scope that transmits said illuminating light for emission from an end of said scope;
a detector that detects the luminance and/or chromaticity of an entering light that enters said scope;
an adjuster that adjusts the light amount and/or white balance of said illuminating light, based on the luminance and/or chromaticity of said entering light; and
a tool in which said end of said scope is inserted, said tool comprising a reflecting member that reflects said illuminating light emitted from said end of said scope so as to become said entering light, the distance between said end of said scope that is inserted in said tool and said reflecting member being adjustable.

2. The endoscope system according to claim 1, wherein said tool further comprises a distance detector that detects said distance and a distance adjuster that adjusts said distance.

3. The endoscope system according to claim 1, wherein said adjuster adjusts the light amount of said illuminating light to a target light amount.

4. The endoscope system according to claim 1, further comprising a memory in which the data representing a target distance that is a target value of the distance between said end of said scope that is inserted in said tool and said reflecting member, is stored.

5. The endoscope system according to claim 1, wherein one of a plurality of said scopes can be selectively used, and further comprises an identifier that identifies said scope that is in use.

6. The endoscope system according to claim 1, wherein one of a plurality of said scopes can be selectively used, a target distance that is a target value of the distance between said end of said scope that is inserted in said tool and said reflecting member, is set for each of said scopes.

7. The endoscope system according to claim 1, further comprising a processor to which said scope is connected, said tool being provided in said processor.

8. The endoscope system according to claim 1, wherein said adjuster comprises a focusing lens that is movable in the direction of the optical axis thereof.

9. The endoscope system according to claim 1, further comprising an adjustment commander that commands said adjuster to adjust the light amount and white balance of said illuminating light in a single operation.

10. A tool, that is used with an endoscope, in which an illuminating light emitted by a light source is transmitted to a subject by a scope, the luminance and/or chromaticity of an entering light that enters said scope is detected, the light amount and/or white balance of said illuminating light is adjustable based on the luminance and/or chromaticity of said entering light, said tool comprising:
an attachment to which said scope is attached; and
a reflecting member that reflects said illuminating light emitted from an end of said scope so as to become said entering light, the distance between said end of said scope that is attached to said attachment and said reflecting member being adjustable.

11. The tool according to claim 10, further comprising a moving member that moves at least one of said attachment and said reflecting member so as to adjust the distance between said end of said scope and said reflecting member.

12. The tool according to claim 10, further comprising a receiver that receives data representing a target distance that is a target value of the distance between said end of said scope that is attached to said attachment and said reflecting member, said data being transmitted by said endoscope.

13. The tool according to claim 10, further comprising a mark that represents relative position of said attachment with respect to said reflecting member, and that is provided on said attachment or said reflecting member.
